(19) United States
(12) Patent Application Publication
(10) Pub. No.: US 2025/0182890 A1
(43) Pub. Date: Jun. 5, 2025

(54) METABOLIC DYSFUNCTION-ASSOCIATED STEATOHEPATITIS BIOMARKER COMPOSITIONS AND APPLICATIONS THEREOF

(71) Applicant: THE CHINESE UNIVERSITY OF HONG KONG SHENZHEN RESEARCH INSTITUTE, Shenzhen, Guangdong (CN)

(72) Inventors: Jun YU, Shenzhen, Guangdong (CN); Wai-Sun Vincent WONG, Shenzhen, Guangdong (CN); Xiang ZHANG, Shenzhen, Guangdong (CN); Guijun ZHAO, Hohhot, Inner Mongolia (CN)

(21) Appl. No.: 18/836,865

(22) PCT Filed: Feb. 10, 2023
(86) PCT No.: PCT/CN2023/075430

§ 371 Date: Aug. 8, 2024

(30) Foreign Application Priority Data
Feb. 10, 2022 (CN) 202210123907.1

(51) Int. Cl. G16H 50/20; G01N 33/68; G16H 50/70

(52) U.S. Cl. CPC G16H 50/20; G01N 33/6893; G16H 50/70; G01N 2800/085

(57) ABSTRACT

A MASH biomarker composition and an application thereof is provided, the biomarker composition including a protein marker and/or clinical biochemical marker for the diagnosis of MASLD and/or MASH, the protein marker being selected from CXCL10, CK-18, P62/SQSTM1, CA3, SQLE, Pro-C3 or one or more of FGF21; the clinical biochemical marker is selected from one or more of BMI, HDL-C, HbA1c, ALT, AST, LDL-C, TG, TC, ALP, or PLT. Biomarker compositions were established by logistic regression and artificial intelligence methods as diagnostic markers for the diagnosis of MASLD and MASH with high sensitivity, specificity, positive predictive value, and negative predictive value in the diagnosis of MASH, independent of age, gender, or metabolic status.

### CROSS-REFERENCE TO RELATED APPLICATIONS

This application is a national stage of PCT/CN2023/075430, filed on Feb. 10, 2023, which claims priority to Chinese Application No. 202210123907.1, filed on Feb. 10, 2022, the entire contents both of which are hereby incorporated by reference.

### FIELD OF TECHNOLOGY

The following relates to the field of bioassays and specifically to a metabolic dysfunction-associated steatohepatitis biomarker composition and its application.

### BACKGROUND

Metabolic dysfunction-associated steatotic liver disease (MASLD) is strongly associated with obesity, type 2 diabetes mellitus, and metabolic syndrome. Due to the prevalence of sedentary lifestyles and high-calorie diets, the incidence of MASLD has shown a rising trend year by year, and has become the most common liver disease in the world, with an average incidence of 15%-40% in Asia, and a higher incidence of 43.3% in the urban areas of China. The pathologic progression of MASLD ranges from simple steatosis to metabolic dysfunction-associated steatohepatitis (MASH), leading to progressive liver fibrosis/cirrhosis and ultimately to liver cancer. 12%-40% of MASLD patients will progress to MASH, 15%-33% of MASH patients will develop cirrhosis, and 15%-27% of cirrhosis patients will progress to liver cancer. The incidence of MASH will increase by 56% in the next decade due to the rising prevalence of obesity and type 2 diabetes. The rate of progression to cirrhosis and hepatocellular carcinoma is significantly higher in patients with MASH than in patients with simple steatosis. Recent studies have shown that there is no effective treatment for MASH-associated hepatocellular carcinoma compared to other causes of hepatocellular carcinoma. The methods to differentiate between simple steatosis and MASH is crucial to prevent disease progression.

The disease process of simple steatosis→MASH→liver fibrosis/cirrhosis→hepatocellular carcinoma is mostly insidious, and liver biopsy has been considered as the “gold” standard for MASH diagnosis. However, the application of liver biopsy is limited in clinical use as it is an invasive test, which is costly and has low acceptance by patients. Therefore, there is an urgent need to establish highly sensitive and specific non-invasive diagnostic methods to reduce the dependence on liver biopsy and to differentiate patients who need further treatment. However, the development of diagnostic markers for MASH is seriously lagging behind, and there are no clinically available methods for non-invasive diagnosis of MASH and prognostic monitoring. A number of diagnostic markers has been used to diagnose MASH non-invasively with low diagnostic accuracy. For example, the apoptosis marker cytokeratin 18 (CK-18) fragment is the most frequently validated blood marker. However, CK-18 alone has a sensitivity of only 66% and a specificity of 82% in MASH diagnosis: The adipocyte factors adiponectin, leptin, and fibroblast growth factor (FGF21), are only associated with metabolic abnormalities in the body and most of them have been validated only in those who have undergone bariatric surgery. Lysosomal enzymes such as Cathepsin D, which varies greatly in different populations, have failed to be validated in external cohorts. Due to the limited accuracy of single factors in MASH diagnosis, there is an urgent need to establish marker combinations to improve the accuracy of diagnosis.

Liver fibrosis is the most important predictor of adverse clinical outcomes in the liver for high-risk MASH. FDA has recommended that drug development be focused on MASH with hepatic fibrosis, an area of significant health need and potential benefit. The development of non-invasive diagnostic markers and kits that can diagnose MASH with hepatic fibrosis in high-risk patients will be important in identifying patients in need of pharmacologic interventions and in monitoring the efficacy of clinical medications.

### SUMMARY

An aspect relates to a biomarker composition that shows high sensitivity, high specificity, high positive predictive value, and high negative predictive value in distinguishing MASH from simple steatosis.

MASH described in embodiments of the present invention can be classified using three different criteria, including:
Type I: steatosis score >=1, inflammation score >=1, ballooning score >=1.
Type II: steatosis score >=1, inflammation score >=1, ballooning score >=1 or NAS score >=4 as defined by the Metabolic dysfunction-associated steatohepatitis Research Network (MASH CRN). The use of the second delineation criterion identifies a greater number of MASH, and therefore can be defined as Boardline MASH.
Type III: High-risk MASH with hepatic fibrosis >=2 in generalized MASH, the delineation criteria were: steatosis score >=1, inflammation score >=1, ballooning score >=1 or NAS score >=4 as defined by the MASH CRN and hepatic fibrosis >=2.

Another aspect of embodiments of the present invention is to provide MASLD diagnostic models and/or MASH diagnostic models. The MASH diagnostic model of embodiments of the present invention is also capable of comparing high and low risk of MASH.

Another aspect of embodiments of the present invention is to provide artificial intelligence models capable of diagnosing MASLD and/or MASH based on the level of the biomarker compositions in the sample to be tested.

Another aspect of embodiments of the present invention is to provide a kit for diagnosing MASH.

In order to solve the above technical problems, embodiments of the present invention adopt the following technical solutions:

A biomarker composition of protein biomarkers for diagnosing MASLD and/or MASH were selected from CXC chemokine ligand 10 (CXCL10), cytokeratin 18 (CK-18), autophagy protein (P62/SQSTM1), carbonic anhydrase III (CA3), squalene epoxygenase (SQLE), collagen type III/IV precursor (Pro-C3), or fibroblast growth factor (FGF21); and clinical biochemical markers are selected from the following: height and weight index (BMI), glycated hemoglobin (HbA1c), alanine aminotransferase (ALT), aminotransferase (AST), low-density lipoprotein cholesterol (LDL-C), total cholesterol (TC), triglycerides (TG), serum alkaline phosphatase (ALP), or platelets (PLT).

In an embodiment, the biomarker composition comprises at least two of the protein markers relevant for diagnosing MASLD and/or MASH and/or high risk MASH.

In an embodiment, the levels of the diagnostic MASLD and/or MASH and/or high-risk MASH related protein markers and the levels of the clinical biochemical markers from the test subject are measured, respectively, and the optimal marker compositions are then established using support vector machines, logistic regression, plain Bayes, and 10-fold cross-validation.

In an embodiment, the biomarker composition comprises five protein markers and four clinical biochemical markers (referred to as N9-MASH) comprising CXCL10, CK-18, P62/SQSTM1, ALT, SQLE, HbA1c, FGF21, PLT and LDL-C.

N9-MASH was able to distinguish patients with metabolic dysfunction-associated steatotic liver disease (MASLD) from healthy individuals, and was further able to distinguish metabolic dysfunction-associated steatohepatitis (steatosis score >=1, inflammation score >=1, ballooning score >=1) from simple steatosis in patients with MASLD.

In an embodiment, the biomarker composition comprises 3 protein markers and 2 clinical biochemical markers (referred to as N5-MASH) comprising CXCL10, CK-18, Pro-C3, AST and BMI.

N5-MASH was able to further distinguish generalized nonalcoholic steatohepatitis (steatosis score >=1, inflammation score >=1, ballooning score >=1 or NAS >=4) from simple steatosis in patients with MASLD.

In an embodiment, the biomarker composition (referred to as N3-MASH) comprises CXCL10, CK-18 and BMI.

N3-MASH was likewise able to further distinguish generalized MASLD (steatosis score >=1, inflammation score >=1, ballooning score >=1 or NAS >=4) from simple steatosis in patients with MASLD.

In an embodiment, the biomarker composition (referred to as N2-Fibrosis) comprises CXCL10 and Pro-C3.

N2-Fibrosis was able to further differentiate high-risk patients with fibrosis in broadly defined MASH.

An application of a biomarker composition as described above in differentiating between MASH and simple steatosis.

A diagnostic model for MASLD and/or a diagnostic model for MASH, constructed by collecting blood samples from patients with MASLD (including patients with MASH and patients with simple steatosis, wherein the patients with MASH include patients with high-risk and low-risk MASH), respectively, and measuring the levels of diagnostic MASH and/or levels of protein markers associated with MASH and/or high-risk MASH and/or levels of clinical biochemical markers, combining the levels of the markers of the patients with MASH and of the healthy persons to build a diagnostic model of MASH using support vector machines, logistic regression, plain Bayes and 10-fold cross validation, and/or, combining the levels of the markers of the patients with MASH and simple steatosis patients with levels of markers to build a diagnostic model for MASH using support vector machines, logistic regression, plain Bayes, and 10-fold cross-validation, and/or, experimental logistic regression of patients with high-risk and low-risk MASH to build a model for diagnosing high-risk MASH patients.

Wherein the protein marker associated with the diagnosis of MASLD and/or MASH is selected from one or more of CXCL10, CK-18, P62/SQSTM1, SQLE, CA3, Pro-C3, or FGF21; and the clinical biochemical marker is selected from HbA1c, AST, BMI, ALT, LDL-C, TG, TC, ALP, or PLT one or more of.

In an embodiment, the biomarker composition used in the diagnostic model of MASLD is N9-MASH.

In an embodiment, the biomarker compositions used in the diagnostic model of MASH are N9-MASH, N5-MASH, N3-MASH or N2-Fibrosis.

An artificial intelligence model for diagnosing MASLD and/or an artificial intelligence model for diagnosing MASH based on the levels of the biomarker compositions in a sample to be tested.

A kit comprising an assay reagent for detecting levels of the biomarker composition.

In an embodiment, the kit further comprises a standard, the standard comprising the biomarker composition.

A use of a biomarker composition as described in the preparation of a kit being used to differentiate between a MASLD and a healthy person, comprising an assay reagent for detecting levels of the biomarker composition.

A use of a biomarker composition as described in the preparation of a kit, being used to differentiate between MASH and simple steatosis, comprising an assay reagent for detecting levels of the biomarker composition.

A use of a biomarker composition as described in the preparation of a kit, being used to differentiate between high-risk MASH and low-risk MASH, comprising an assay reagent for detecting levels of the biomarker composition.

Application of the described kits in differentiating between MASLD and healthy individuals, and/or, differentiating between MASH and simple steatosis, and/or, differentiating between high risk MASH and low risk MASH.

In an embodiment, the method of differentiation is as follows: providing a sample derived from the subject to be tested, testing the sample for levels of the relevant protein markers for diagnosing MASLD and/or MASH and the clinical biochemical markers, respectively, and then substituting for support vector machines, logistic regression, plain Bayesian, and 10-fold cross-validation to determine the optimal combination of biomarkers, and performing a ROC curve analysis in a diagnostic model of MASLD to differentiate between MASLD and healthy individuals based on Cut-off values: In the diagnostic model for MASH, the Cut-off value is used to differentiate between MASH and simple steatosis: in the diagnostic model for high-risk MASH, the Cut-off value is used to differentiate between high-risk and low-risk MASH patients.

In an embodiment, the Cut-off value is determined by ROC analysis of maximised Yoden index, the maximized Yoden index=sensitivity+specificity−1.

The Cut-off threshold was used as the positive judgement value, ≥Cut-off threshold was judged as positive, <Cut-off threshold was judged as negative.

When the biomarker composition is N9-MASH, it is desired that the Cut-off threshold in the diagnostic model for MASLD is 0.58; and it is desired that the Cut-off threshold in the diagnostic model for MASH is 0.47 (the models for the diagnosis of MASLD and MASH are different machine learning models).

According to some embodiments, when the biomarker composition is N9-MASH, a diagnostic model of MASLD has the highest sensitivity and specificity for distinguishing between MASLD and a healthy control when the Cut-off threshold value is 0.58, and if the Cut-off value is ≥0.58, MASLD is awarded, and a Cut-off value <0.58 were judged as healthy:

According to some embodiments, when the biomarker composition is N9-MASH, a diagnostic model of MASH performs best in distinguishing between MASH and simple steatosis when the Cut-off threshold value is 0.47, and a diagnosis of MASH is made if the Cut-off value is ≥0.47, and a diagnosis of MASH is made if the Cut-off value is <0.47, simple steatosis was diagnosed.

When the biomarker composition is N5-MASH, it is desired that the Cut-off thresholds in the diagnostic model of MASH are 0.27 and 0.61.

According to some embodiments, when the biomarker composition is N5-MASH, a diagnostic model of MASH is diagnosed with MASH if the Cut-off value is ≥0.61 and simple steatosis if the Cut-off value is <0.27. If 0.27≤Cut-off value <0.61, a liver biopsy was required.

When the biomarker composition is N3-MASH, the Cut-off thresholds in the diagnostic model of MASH are 0.43 and 0.68.

According to some embodiments, when the biomarker composition is N3-MASH, a diagnostic model of MASH is diagnosed with MASH if the Cut-off value is ≥0.68, and simple steatosis if the Cut-off value is <0.43. If 0.43≤Cut-off value <0.68, liver puncture biopsy was required.

When the biomarker composition is N2-Fibrosis, Cut-off thresholds of 0.37 and 0.30 in a diagnostic model of high-risk MASH are desired.

According to some embodiments, when the biomarker composition is N2-Fibrosis, a diagnostic model of high-risk MASH is diagnosed as high-risk MASH requiring tertiary management if the Cut-off value is ≥0.37. If Cut-off value <0.30, the diagnosis was low-risk MASH and only primary lifestyle intervention was required. If the Cut-off value was 0.30≤Cut-off <0.37, a liver puncture biopsy was required.

In an embodiment, the sample is blood.

In an embodiment, the sample is serum.

In an embodiment, the sample is from a human.

Candidate biomarkers of embodiments of the present invention were selected by ranking the importance of protein markers and other clinical variables identified in our previous studies using varImp.

Embodiments of the present invention have the following advantages over the conventional art:

Embodiments of the present invention identify biomarker compositions that allow for the non-invasive diagnosis of MASLD and/or MASH, the biomarker compositions show high sensitivity, high specificity, high positive predictive value (PPV), and high negative predictive value (NPV) in differentiating between MASH and simple steatosis, and the biomarker compositions of embodiments of the present invention are capable of differentiating between high-risk and low-risk MASH from other MASLD. The biomarker compositions of embodiments of the present invention can be assayed by widely used methods and can be readily applied in clinical practice. The diagnostic performance of the biomarker compositions of embodiments of the present invention is not affected by age, gender or metabolic status.

### BRIEF DESCRIPTION

Some of the embodiments will be described in detail, with references to the following Figures, wherein like designations denote like members, wherein:

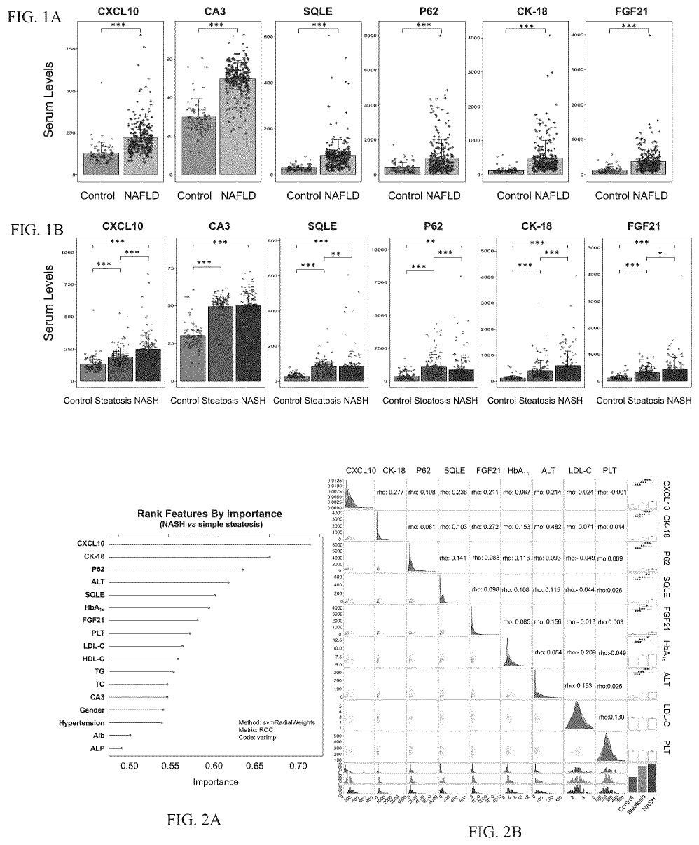

FIG. 1A shows a graph of CXCL10, SQLE, CK-18, P62/SQSTM1, FGF21, and CA3 levels in the serum of MASLD patients compared with healthy controls:

FIG. 1B shows the comparison of serum levels of CXCL10, SQLE, CK-18, P62/SQSTM1, FGF21 and CA3 in MASH patients and healthy controls;

FIG. 2 shows the ranking of the importance of the 17 markers for the diagnosis of MASH:

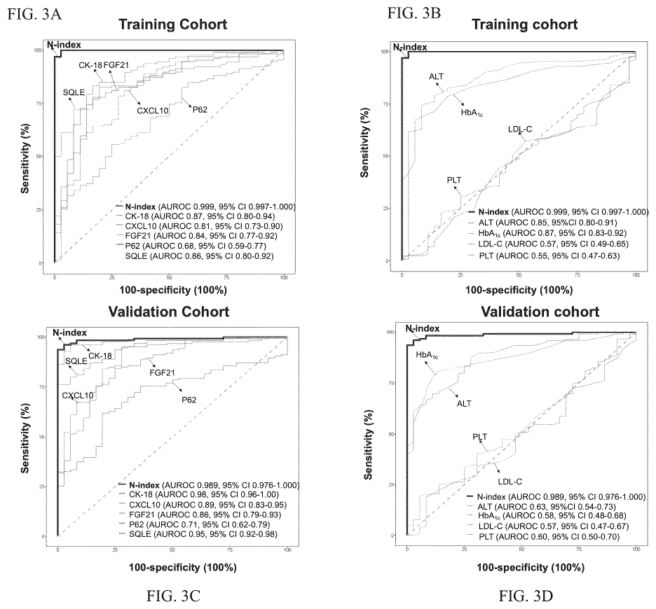

FIG. 3A shows a comparison of the performance of N9-MASH and individual biomarkers for the diagnosis of MASH in healthy controls and for the diagnosis of MASLD, as well as in patients with MASLD:

FIG. 3B shows a comparison of the performance of N9-MASH and individual biomarkers for the diagnosis of MASH in healthy controls and for the diagnosis of MASLD, as well as in patients with MASLD:

FIG. 3C shows a comparison of the performance of N9-MASH and individual biomarkers for the diagnosis of MASH in healthy controls and for the diagnosis of MASLD, as well as in patients with MASLD:

FIG. 3D shows a comparison of the performance of N9-MASH and individual biomarkers for the diagnosis of MASH in healthy controls and for the diagnosis of MASLD, as well as in patients with MASLD:

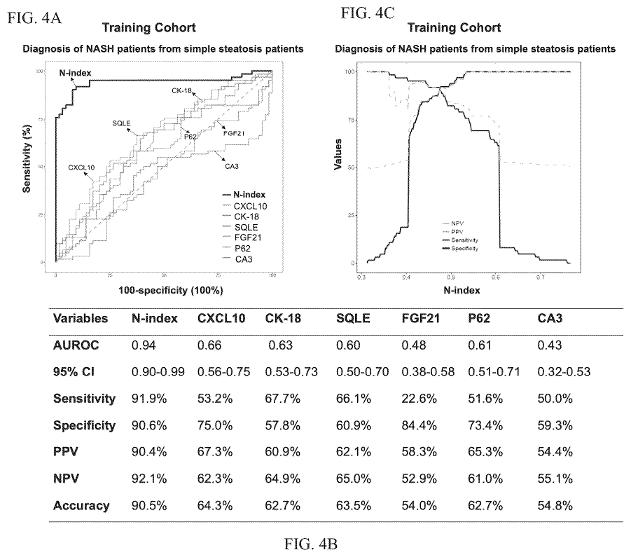

FIG. 4A shows a graph comparing the performance of N9-MASH and individual biomarkers for MASH diagnosis in the training cohort:

FIG. 4B shows a graph comparing the performance of N9-MASH and individual biomarkers for MASH diagnosis in the training cohort:

FIG. 4C shows a graph comparing the performance of N9-MASH and individual biomarkers for MASH diagnosis in the training cohort:

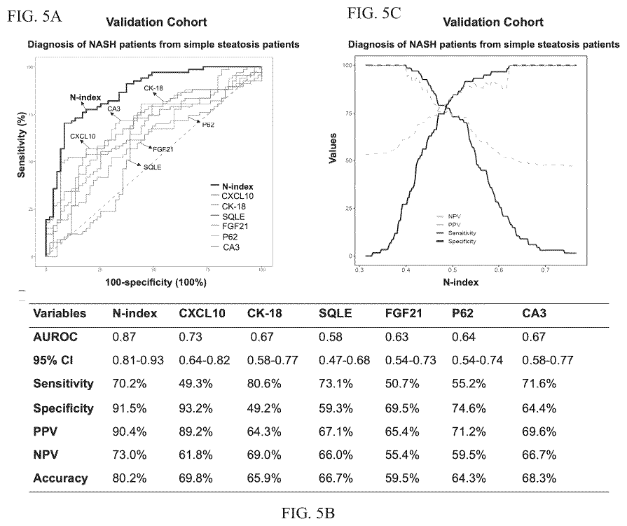

FIG. 5A shows a graph comparing the performance of N9-MASH and individual biomarkers for the diagnosis of MASH in the validation cohort:

FIG. 5B shows a graph comparing the performance of N9-MASH and individual biomarkers for the diagnosis of MASH in the validation cohort:

FIG. 5C shows a graph comparing the performance of N9-MASH and individual biomarkers for the diagnosis of MASH in the validation cohort:

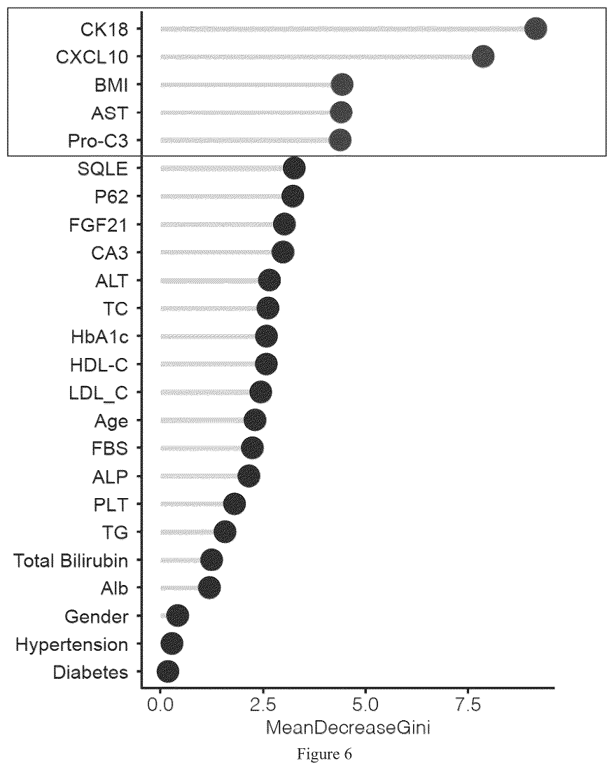

FIG. 6 shows the ranking of the importance of markers for the diagnosis of MASH in the broad sense:

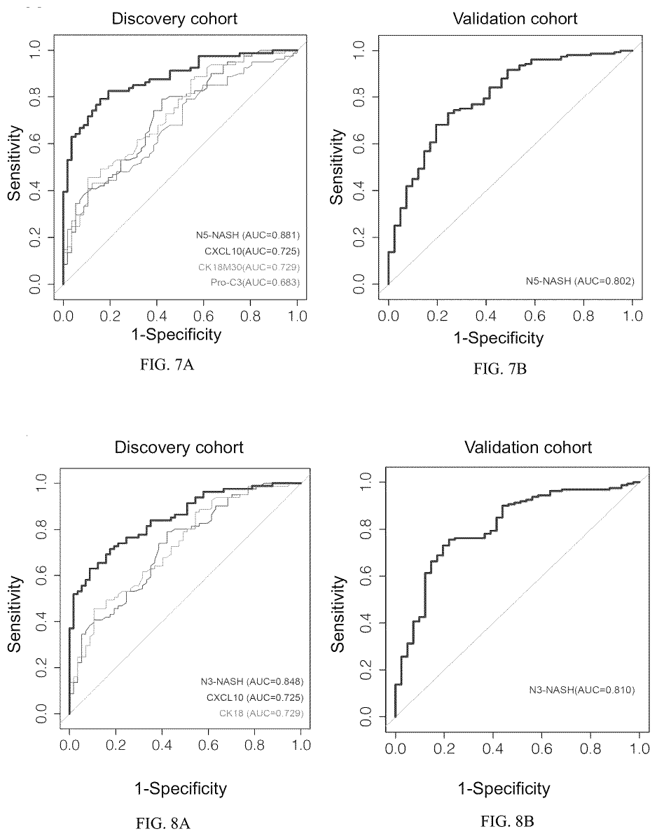

FIG. 7A shows the performance of N5-MASH for MASH diagnostics in the discovery and verification queues:

FIG. 7B shows the performance of N5-MASH for MASH diagnostics in the discovery and verification queues:

FIG. 8A shows the performance of N3-MASH for MASH diagnostics in the discovery and verification queues:

FIG. 8B shows shows the performance of N3-MASH for MASH diagnostics in the discovery and verification queues; and

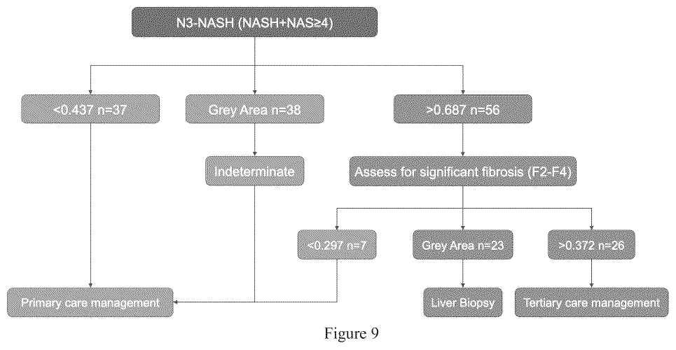

FIG. 9 shows the N3-MASH and N2-Fibrosis two-step method for diagnosing high-risk MASH.

### DETAILED DESCRIPTION

Embodiments of the present invention are not limited to the following embodiments. The implementation conditions adopted in the embodiments can be further adjusted according to the different requirements of specific use, and the unspecified implementation conditions are the conventional conditions in this industry. The technical features involved in the various embodiments of the present invention can be combined with each other as long as they do not conflict with each other.

In order to improve the accuracy of non-invasive MASH diagnosis, the inventors identified a number of protein biomarkers associated with MASH after preliminary work, and combined them with clinical biochemical indicators capable of identifying patients with MASLD and MASH, introducing 24 variables, including 7 serum proteins (CXCL10 SQLE, CK-18 P62/SQSTM1 FGF21 Pro-C3 and CA3) and 17 clinical variables (14 continuous variables: (age body mass height index (BMI) platelets (PLT) serum albumin (Alb), serum alkaline phosphatase level (ALP) serum alanine aminotransferase (ALT), azelaic acid transaminase (AST) bilirubin (bilirubin), total cholesterol (TC), high density lipoprotein cholesterol (HDL-C) triglycerides (TG) low-density lipoprotein cholesterol (LDL-C) glycated haemoglobin (HbA1c)) fasting blood sugar (FBS) and 3 discrete variables (gender, hypertension diabetes)) were used to train the marker model Embodiments of the present invention identify optimal diagnostic biomarker compositions for MASH.

### Study Subject:

A total of 374 subjects were recruited from the Prince of Wales Hospital in Hong Kong. After excluding 36 subjects with other causes of liver injury and 14 subjects with insufficient serum samples. 324 subjects including 252 patients with biopsy-confirmed MASLD and 72 healthy controls participated in the study. Liver biopsy is performed in patients with MASLD primarily due to their abnormal liver function tests and/or abnormal imaging results. Exclusion criteria included 1) drinking more than 30 g of alcohol per day for men and more than 20 g for women: 2) hepatitis B surface antigen or anti-hepatitis C virus antibody positivity with antinuclear antibody titre > 1/160; and 3) patients with secondary hepatic steatosis or other histological features of liver disease. Control subjects were randomly selected from a government census database. Proton magnetic resonance spectroscopy (1H-MRS) was performed to quantify hepatic triglyceride levels in subjects who agreed to participate in the study. Control subjects were excluded if 1) hepatic triglyceride levels exceeded 5%. 2) history of diabetes or hypertension, and 3) patients with MASLD were excluded. Fasting venous blood samples were collected the day before liver biopsy. All patients provided written informed consent to participate in the trial and to collect blood samples specifically for biomarker studies.

Independent validation cohort specimens were obtained from 217 patients with MASLD confirmed by liver puncture, and liver pathology was taken from ultrasound-guided percutaneous liver biopsies using either 16G or 18G needles. A total of 201 patients with MASLD were included after excluding subjects with unexplained histopathology as well as other causes of liver injury. MASLD activity scores (NAS) was obtained by calculating the steatosis score+inflammation score+ballooning score in Pathology. Liver fibrosis is defined as: grade 0), no fibrosis: grade 1, perisinusoidal or portal fibrosis: grade 2, perisinusoidal/periportal fibrosis; grade 3, bridging fibrosis; and grade 4, cirrhosis.

### Diagnostic Criteria:

1. MASH: Liver steatosis score >=1, inflammation score >=1, ballooning score >=1.
2. Broad MASH: In addition to the MASH shown in 1), includes NAS scores >=4 as defined by the Nonalcoholic Steatohepatitis Research Network (MASH CRN).
3. High-risk MASH: Liver fibrosis score >=2 in broad MASH.

### Serum Protein Test:

Serum was collected from −80° C. for biomarker assay. Serum CK-18, FGF21, CXCL10, P62/SQSTM1, SQLE, CA3 serum levels were measured by M30 enzyme-linked immunosorbent assay (ELISA) kit (PEVIVA, Sweden), FGF21 ELISA kit (BioVendor, Czech Republic), CXCL10 ELISA kit (R&D), P62/SQSTM1 ELISA kit (Cloud-Clone, USA), SQLE ELISA kit (Cloud-Clone, USA), Pro-C3 ELISA kit (Nordic Bioscience, Denmark) and CA3 ELISA kit (Cloud Clone, USA) were tested.

### Algorithm Training

We introduced 24 variables including 7 serum proteins (CXCL10, SQLE, CK-18, P62/SQSTM1, FGF21, Pro-C3 and CA3) and 17 clinical variables to train the marker model. Clinical variables included 14 continuous variables (age, body mass height index (BMI), platelets (PLT), serum albumin (Alb), serum alkaline phosphatase level (ALP), serum alanine aminotransferase (ALT), azelaic transaminase (AST), bilirubin (bilirubin), total cholesterol (TC), high-density lipoprotein cholesterol (HDL-C), triglycerides (TG), low-density lipoprotein cholesterol (LDL-C), glycosylated hemoglobin (HbA1c), and fasting blood sugar (FBS)) and three discrete variables (gender, hypertension, and diabetes). Correlation analyses were performed for covariates with maximum correlation (absolute value of Spearman's correlation coefficient, abs (rho) ≥0.5). The importance of each variable is ordered by varImp. Selection of key biomarkers and clinical variables by recursive feature elimination. Optimal diagnostic biomarkers for MASH were identified by using support vector machines, neural networks, plain Bayesian logistic regression, and 10-fold cross-validation to build a diagnostic model that examined marker selection criteria via the Wilcoxon rank sum test.

Diagnostic performance of biomarker combinations was assessed using AUROC analysis. The optimal cut-off value was determined by ROC analysis maximizing the Jordon index (J=sensitivity+specificity−1).

### Statistical Analysis

Proxy variable analysis (sva) was used in data preprocessing in order to eliminate batch effects. Values in the experiment are expressed as mean±standard deviation (SD). Differences in protein levels or clinical variables were determined by the Mann-Whitney U test. a P value of 0.05 was considered statistically significant. Spearman's correlation coefficient was applied to assess the association of biomarker levels with relevant factors and was used in multiple regression analysis to identify independent factors associated with biomarkers. DeLong's test was used to compare the AUROC values of different subgroups.

### Implementation Example I: Biomarkers of MASH

To identify candidate biomarkers that could be diagnostic of MASLD and MASH, we examined serum levels of CXCL10, SQLE, CK-18, P62/SQSTM1, FGF21, and CA3 in 252 MASLD patients and 72 healthy controls. Of the 252 MASLD patients recruited, 129 (51%) had a diagnosis of MASH confirmed by pathologic evaluation (hepatic steatosis score >=1, inflammation score >=1, and ballooning score >=1). Serum levels of all six biomarkers were significantly higher in MASLD patients compared with healthy controls (P<0.0001, FIG. 1A). In patients with MASLD, serum CXCL10, SQLE, CK-18, and FGF21 levels were significantly higher in patients with MASH (n=129) than in patients with simple steatosis (n=123) (FIG. 1B).

CXCL10 and CK-18 were positively associated with lobular inflammation (CXCL10, rho: 0.21, P<0.001; CK-18, rho: 0.21, P=0.001) and hepatocellular ballooning-like changes (CXCL10, rho: 0.26, P<0.0001: CK-18 in MASLD patients, rho: 0.27, P<0.0001) were positively correlated, while SQLE was positively correlated with inflammation (rho: 0.20, P<0.001).

We further analyzed clinical variables that may serve as candidate biomarkers for the diagnosis of MASLD and MASH. Among the 17 clinical variables included were age, sex, height, weight, waist circumference, hypertension, diabetes mellitus, platelets, serum alanine aminotransferase, glutamic oxalate aminotransferase, alkaline phosphatase, albumin, total cholesterol, total triglycerides, LDL cholesterol, HDL cholesterol, and glycosylated hemoglobin. HbA1c (an indicator of glucose metabolism) and ALT (an indicator of liver injury) increased progressively in controls, patients with simple steatosis, and patients with MASH.

We selected the most appropriate biomarkers for the diagnosis of MASLD and MASH by varImp ranking all markers by importance. CXCL10, CK-18, P62/SQSTM1, ALT, SQLE. HbA1c, FGF21, PLT, and LDL-C were the top 9 most important variables for the diagnosis of MASH (FIG. 2A). In addition, there was no strong correlation between these nine variables in all subjects (|rho|: 0.001-0.482), suggesting that these markers are indispensable to each other (FIG. 2B). Therefore, we used these nine independent biomarkers, including five MASH-associated proteins (CXCL10, CK-18, P62/SQSTM1, SQLE, and FGF21) and four blood biochemical variables (HbA1c, ALT, LDL-C, and PLT) to develop a MASH diagnostic model. SVM was used to train the model and reduce model overfitting or selection bias. The optimal biomarker combination was eventually established and called N9-MASH, including the first 9 variables mentioned above.

We evaluated the performance of N9-MASH on healthy controls and in terms of MASLD diagnosis. N9-MASH distinguished MASLD patients from healthy controls with an AUROC of 0.999 (95% CI 0.997-1.000) (FIG. 3A), and the performance of N9-MASH was superior to that of individual protein biomarkers (AUROC 0.68-0.87, all P<0.01) or clinical variables (AUROC 0.55-0.87, all P<0.0001) was superior (FIG. 3B). The sensitivity and specificity of N9-MASH for the diagnosis of MASLD were 100% and 97.2%, respectively.

We then evaluated the performance of N9-MASH in diagnosing MASH in patients with MASLD. Among MASLD patients, MASH patients could be identified by N9-MASH with an AUROC of 0.94 (95% CI 0.90-0.99) (FIG. 4A). N9-MASH had significantly higher accuracy in distinguishing patients with MASH from those with simple steatosis compared to individual protein markers: CXCL10 (AUROC 0.66, 95% CI 0.56-0.75, P<0.0001), CK-18 (AUROC 0.63, 95% CI 0.53-0.73, P<0.0001), SQLE (AUROC 0.60, 95% CI 0.50-0.70, P<0.0001), FGF21 (AUROC 0.48, 95% CI 0.38-0.58, P<0.0001), CA3 (AUROC 0.43, 95% CI 0.32-0.53, P<0.0001), or P62/SQSTM1 (AUROC 0.61, 95% CI 0.51-0.71, P<0.0001) (FIG. 4A-B): or individual clinical variables (AUROC 0.52-0.63, all P<0.0001).

The sensitivity, specificity, NPV, and PPV of N9-MASH for the diagnosis of MASH in the training cohort are shown in FIG. 4C. At 90.6% specificity, N9-MASH had 91.9% sensitivity, 90.5% accuracy, 92.1% NPV, and 90.4% PPV (FIG. 4B).

Similar to the training cohort, N9-MASH performed well in distinguishing MASLD patients from healthy controls with an AUROC of 0.989 (95% CI 0.976-1.000) (FIG. 3C-D). At a specificity of 100%, N9-MASH had a sensitivity of 93.7% and an accuracy of 95.1% in the diagnosis of MASLD.

For MASH diagnosis, N9-MASH was significantly better than individual protein markers (FIG. 5A-B) or clinical variables. FIG. 5C illustrates the sensitivity, specificity, NPV, and PPV of N9-MASH in differentiating MASH patients from the validation cohort at different thresholds. At 91.5% specificity, N9-MASH showed 70.2% sensitivity, 80.2% accuracy, 73.0% NPV, and 90.4% PPV in distinguishing patients with MASH from those with simple steatosis, which was significantly higher than that of the individual biomarkers (FIG. 5B). Thus, these results confirm the diagnostic value of N9-MASH for MASH.

In summary, embodiments of the present invention identify the optimal biomarker combination for the diagnosis of MASLD and MASH and is referred to as N9-MASH, comprising five protein markers (CXCL10, CK-18, P62/SQSTM1, SQLE, and FGF21) and four blood biochemical variables (HbA1c, ALT, LDL-cholesterol, and platelets).

N9-MASH can distinguish MASLD patients from healthy controls this, with AUROC diagnostic accuracy up to 0.999 in the training cohort and 0.989 in the validation cohort.

Among MASLD patients, MASH patients could be recognized by N9-MASH with an AUROC of 0.94 in the training cohort and 0.87 in the validation cohort.

N9-MASH had 90.6% specificity, 91.9% sensitivity and 90.4% positive predictive value (PPV) for distinguishing MASH patients from simple steatosis in the training cohort, and its diagnostic performance was equally good in the validation cohort (91.5% specificity, 70.2% sensitivity and 90.4% PPV).

In the diagnostic model of MASLD, the Cut-off critical value of 0.58 had the highest sensitivity and specificity for distinguishing MASLD from healthy controls, and if the Cut-off value was ≥0.58, MASLD was adjudicated as MASLD, and Cut-off value <0.58 was adjudicated as healthy:

In the diagnostic model for MASH, the best performance in distinguishing between MASH and simple steatosis was achieved at a Cut-off critical value of 0.47, with a diagnosis of MASH if the Cut-off value was >0.47, and a diagnosis of simple steatosis if the Cut-off value was <0.47.

### Implementation Example II: Biomarkers of Broadly Defined MASH

Clinical trials for MASH typically include patients with NAS>=4. Further, we analyzed markers that could distinguish broadly defined MASH (hepatic steatosis score >=1, inflammation score >=1, balloon score >=1, or NAS>=4). We included 145 MASLD samples from the Hong Kong cohort and excluded 7 cases with unexplained pathological diagnoses, resulting in 138 MASLD patients included in the discovery cohort, of which 81 were diagnosed with generalized MASH and 57 with simple steatosis based on pathological findings. Another cohort of 201 samples was included as an independent validation cohort, of which 160 had generalized MASH and 41 had simple steatosis.

The marker model was trained by analyzing 24 variables including 7 serum proteins (CXCL10, SQLE, CK-18, P62/SQSTM1, FGF21, Pro-C3, and CA3) and 17 clinical variables (14 continuous variables (age, BMI, PLT, Alb, ALP, ALT, AST, TC, Bilirubin, HDL-C. TG. LDL-C, HbA1c, FBS) and 3 discrete variables (gender, hypertension, diabetes)) to train the marker model. Important variables were screened by Random Forest approach (FIG. 6) and modeled by Park's Bayesian and logistic regression methods, resulting in the optimal combination of biomarkers, termed N5-MASH, including CXCL10, CK-18, Pro-C3, AST, and BMI. In the logistic regression model,

N5-MASH=2.655*CK-18+3.06*CXCL10-0.066*Pro-C3-0.0802*BMI-0.076*AST-1.62.

In the discovery queue, N5-MASH distinguishes the AUROC in generalized MASH as 0.881 (FIG. 7A). The accuracy of N5-MASH in distinguishing patients with generalized MASH from those with simple steatosis was significantly higher compared to individual protein markers: CXCL10 (AUROC 0.725, P<0.001), CK-18 (AUROC 0.719, P<0.001), or Pro-C3 (AUROC 0.683, P<0.0001) (FIG. 7A-B). In the validation queue, N5-MASH can also significantly distinguish generalized MASH (AUROC=0.802, FIG. 7B).

When the described biomarker compositions are N5-MASH, the diagnostic model of MASH with Cut-off critical values of 0.27 and 0.61 allows for the diagnosis of MASH with high specificity (>90%) and high sensitivity (>90%), respectively, and the diagnosis of MASH is made if the Cut-off value is >0.61, and the diagnosis of MASH is made if the Cut-off value <0.27, simple steatosis was diagnosed. With a Cut-off threshold value of 0.61, N3-MASH had a specificity of 91.2%, a sensitivity of 67.9% and a PPV of 91.7% in diagnosing generalized MASH. With a low Cut-off critical value of 0.27, N3-MASH had a sensitivity of 90.1%, a specificity of 54.4%, and a NPV of 79.5% in ruling out generalized MASH, and 38 patients (27.5%) were in the intermediate region, requiring further liver biopsy.

To increase the feasibility of clinical application, we further optimized the marker model to investigate whether similar diagnostic efficiency could be achieved by applying a small number of markers. We found that the model combination of CXCL10, CK-18, and BMI (N3-MASH) could diagnose generalized MASH with high sensitivity and specificity (discovery cohort: AUROC-0.848: validation cohort: AUROC=0.810) (FIG. 8). The logistic regression model is as follows: N3-MASH=2.75*CK-18+3.42*CXCL10+0.08*BMI-16.47.

With a Cut-off threshold of 0.68, N3-MASH had a specificity of 91.2%, a sensitivity of 62.9%, and a PPV of 91.0% in diagnosing generalized MASH. With a low Cut-off threshold value of 0.43, N3-MASH had a sensitivity of 90.1%, a specificity of 49.1%, and an NPV of 77.8% in excluding generalized MASH. Forty-five patients (32.6%) were in the intermediate region and required further liver biopsy. In the validation cohort, N3-MASH had a specificity of 92.7%, a sensitivity of 38.1%, and a PPV of 95.3% in the diagnosis of generalized MASH at a Cut-off threshold of 0.68. With a low Cut-off threshold of 0.43, N3-MASH had a sensitivity of 65.6%, specificity of 85.4%, NPV of 38.9%, and PPV of 94.5% in ruling out generalized MASH, and 48 patients (23.8%) were in the intermediate region, requiring further liver biopsy testing. Therefore, in practice, a high Cut-off threshold value of 0.68 was chosen to diagnose generalized MASH, and a low Cut-off of 0.43 was chosen to exclude simple steatosis, i.e., when the described biomarker combination was N3-MASH, a diagnosis of MASH was made in the diagnostic model of MASH if the Cut-off value was ≥0.68, requiring a Tertiary care. A diagnosis of simple steatosis was made if the Cut-off value was <0.43, and patients with a Cut-off value of <0.68 required only primary care with lifestyle interventions (FIG. 9).

### Implementation Example III: Biomarkers of High-Risk MASH

To further distinguish high-risk patients with fibrosis in MASLD, we analyzed specimens diagnosed with generalized MASLD. 119 patients with a diagnosis of MASH were included in the study, 56 in the discovery cohort and 63 in the validation cohort.

The marker model was trained by analyzing 24 variables including 7 serum proteins (CXCL10, SQLE, CK-18, P62/SQSTM1, FGF21, Pro-C3, and CA3) and 17 clinical variables (14 continuous variables (age, BMI, PLT, Alb, ALP. ALT, AST, TC, Bilirubin, HDL-C, TG, LDL-C. HbA1c, FBS) and 3 discrete variables (gender, hypertension, diabetes)) to train the marker model. The optimal biomarker combination, termed N2-Fibrosis, was established by logistic regression methods and included CXCL10 and Pro-C3. In this logistic regression model N2-Fibrosis=0.83*CXCL10+0.03*Pro-C3-3.02.

The AUROC for N2-Fibrosis differentiation accompanied by high-risk MASH was 0.784 in the discovery cohort and 0.856 in the validation cohort.

In practice, among patients with N3-MASH Cut-off values higher than 0.68 diagnosed with generalized MASH, those with N2-Fibrosis Cut-off thresholds higher than 0.37 were diagnosed with high-risk MASH and required tertiary management. Patients with Cut-off values between 0.30 and 0.37 required further confirmation by hepatic puncture biopsy. Patients with Cut-off critical values below 0.30 were diagnosed with low-risk MASH and required only primary management with lifestyle interventions (FIG. 9).

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A biomarker composition comprising:
an associated protein marker for diagnosing MASLD and/or MASH, and/or
a clinical biochemical marker;
the associated protein marker for diagnosing MASLD and/or MASH being selected from one or more of: CXCL10, CK-18, P62/SQSTM1, SQLE, CA3, Pro-C3, or FGF21;
wherein the clinical biochemical marker is selected from one or more of BMI, HDL-C, HbA1c, ALT, AST, LDL-C, TG, TC, ALP, or PLT.

2. The biomarker composition according to claim 1, wherein the biomarker composition comprises CXCL10, CK-18, AST, P62/SQSTM1, SQLE, Pro-C3, FGF21, BMI, HbA1c, ALT, LDL-C and PLT.

3. The biomarker composition according to claim 1, wherein the biomarker composition comprises CXCL10, CK-18, P62/SQSTM1, ALT, SQLE, HbA1c, FGF21, PLT and LDL-C.

4. The biomarker composition according to claim 1, wherein the biomarker composition comprises CXCL10, CK-18, Pro-C3, AST and BMI.

5. The biomarker composition according to claim 1, wherein the biomarker composition of comprises CXCL10, CK-18 and BMI.

6. The biomarker composition according to claim 1, wherein the biomarker composition of comprises CXCL10 and Pro-C3.

7. A diagnostic model for MASLD and/or MASH, wherein a blood sample is collected from a patient with MASLD and/or MASH, and levels of MASH-related protein markers and/or clinical biochemical markers are measured, respectively, and the diagnostic model for MASLD and/or MASH is established using Neural Network, Naïve Bayesian, Logistic Regression and 10-fold cross-validation. Neural Network, Naïve Bayesian, Logistic Regression and 10-fold cross validation are used to establish the diagnostic model for MASLD and/or MASLD, and the MASH-related protein markers are selected from one or more of CXCL10, CK-18, P62/SQSTM1, SQLE, CA3, SQLE, CA3, Pro-C3, or FGF21; the clinical biochemical markers are selected from one or more of BMI, HDL-C, HbA1c, ALT, AST, LDL-C, TG, TC, ALP, or PLT.

8. The diagnostic model for A MASLD and/or MASH according to claim 7, wherein it is constructed specifically as follows: collecting a blood sample from a patient with MASLD, measuring respectively the levels of the diagnostic MASLD and/or MASH related protein markers and/or the levels of the clinical biochemical markers, modeling the levels of the markers in patients with MASLD and healthy individuals for the diagnosis of MASLD using support vector machines, logistic regression, plain Bayes, and 10-fold cross-validation, and/or, modeling the levels of the markers in patients with MASH and patients with steatosis simplex using support vector machines, logistic regression, plain Bayes and 10-fold cross-validation to build a diagnostic model for MASH.

9. The diagnostic model for MASLD and/or MASH according to claim 7, wherein a biomarker composition used in the diagnostic model for MASLD comprising CXCL10, CK-18 P62/SQSTM1, ALT, SQLE, HbA1C, FGF21, PLT and LDL-C;
a biomarker composition used in the diagnostic model of MASH comprising CXCL10, CK-18, P62/SQSTM1, ALT, SQLE, HbA1c, FGF21, PLT and LDL-C, or comprising CXCL10, CK-18, Pro-C3, AST and BMI, or comprising CXCL10, CK-18 and BMI.

10. The diagnostic model for MASLD and/or MASH according to claim 7, wherein MASH patient comprises a high-risk and a low-risk MASH patient, and that an experimental logistic regression of the high-risk and low-risk MASH patient is established to establish a high-risk MASH diagnostic model, and a biomarker composition used in the diagnostic model for MASLD comprising CXCL10 and Pro-C3.

11. A biomarker composition according to claim 1 at a level in a sample to be tested to diagnose MASLD and/or MASH by means of a logistic regression or artificial intelligence model built up.

12. A kit for detecting levels of the biomarker composition according to claim 1 comprising an assay reagent for detecting levels of the biomarker composition.

13. The kit according to claim 12, wherein the kit further comprises a standard, the standard comprising the biomarker composition.

14. The kit according to claim 12, wherein the kit being used to differentiate between a MASLD patient and a healthy person, and/or, the kit being used to differentiate between a MASH patient and a simple steatosis patient, and/or, the kit is for differentiating between high risk MASH patient and low risk MASH patient.

15. The kit according to claim 14, wherein a method of differentiation is: providing a sample derived from the subject to be tested, testing the sample for levels of the protein markers associated with diagnosis of MASLD and/or MASH and the clinical biochemical markers, respectively, and then substituting neural network, plain Bayes, logistic regression and 10-fold cross validation to determine the optimal biomarker combination to differentiate between MASLD and/or MASH and/or simple steatosis and/or healthy individuals based on the Cut-off value of the artificial intelligence model, the Cut-off value being determined by a ROC analysis maximizing the Jordon index, maximizing the Jordon index=sensitivity+specificity−1.

16. The kit according to claim 15, wherein the sample is blood.

17. The kit according to claim 15, wherein the sample is from a human.

18. A method for diagnosis of MASLD and/or MASH, wherein in the level of a biomarker combination is tested in a blood sample of a patient, a optimal biomarker combination is determined by substitution of support vector machines, logistic regression, plain Bayesian, and 10-fold cross-validation, and a ROC curve analysis is performed; wherein the biomarker combination CXCL10, CK-18, P62/SQSTM1, ALT, SQLE, HbA1S, FGF21, PLT and LDL-C, or comprising CXCL10, CK-18, Pro-C3, AST and BMI, or comprising CXCL10, CK-18 and BMI;
in a diagnostic model of MASLD, the distinction between MASLD and healthy individuals is based on the Cut-off value, and a diagnosis of MASLD is made if the Cut-off value is ≥the Cut-off threshold, and a diagnosis of healthy individuals is made if the Cut-off value is <the Cut-off threshold;
in a diagnostic model of MASH, MASH and simple steatosis are differentiated according to the Cut-off value, and the Cut-off value includes a low Cut-off threshold value and a high Cut-off threshold value, and if the patient's Cut-off value≥high Cut-off threshold value, MASH is diagnosed, and if the Cut-off value<low Cut-off threshold, simple steatosis was diagnosed, and if low Cut-off threshold≤Cut-off value<high Cut-off threshold, liver puncture biopsy was required;
adopting the biomarker composition comprising CXCL10 and Pro-C3 in a diagnostic model for high-risk MASH, distinguishing high-risk MASH from low-risk MASH based on a Cut-off value, the Cut-off value comprising a low Cut-off threshold and a high Cut-off threshold, if the patient has a Cut-off value≥high Cut-off threshold, then a diagnosis of high-risk MASH is made, if the Cut-off value<low Cut-off threshold, then a diagnosis of low-risk MASH is made, and if the low Cut-off threshold≤Cut-off value<high Cut-off threshold, then a liver biopsy is required.

19. The method for the diagnosis according to claim 18, wherein when the biomarker composition is the biomarker composition in the diagnostic model for MASLD, the Cut-off threshold is 0.58, and if the Cut-off value is ≥0.58, the diagnosis is made as MASLD, and the Cut-off value is <0.58 judged as a healthy person; in the diagnostic model of MASH, Cut-off threshold is 0.47, if Cut-off value≥0.47, then diagnosed as MASH, if Cut-off value<0.47, then diagnosed as simple steatosis:
when the biomarker composition is the biomarker composition in the diagnostic model for MASH has a low Cut-off threshold of 0.27 and a high Cut-off threshold of 0.61, and if the Cut-off value is ≥0.61, the diagnosis is MASH, and if the Cut-off value is <0.27, the diagnosis is simple steatosis, and if 0.27≤Cut-off value <0.61, a hepatic puncture biopsy was required;
when the biomarker composition is the biomarker composition in the diagnostic model for MASH has a low Cut-off threshold of 0.43 and a high Cut-off threshold of 0.68, and if the Cut-off value is ≥0.68, the diagnosis is MASH, and if the Cut-off value is <0.43, the diagnosis is simple steatosis, and if 0.43≤Cut-off value<0.68, a hepatic puncture biopsy was required;
when the biomarker composition is the biomarker composition in the diagnostic model for high-risk MASH has a low Cut-off threshold of 0.30 and a high Cut-off threshold of 0.37, and if the Cut-off value is ≥0.37, then the diagnosis is high-risk MASH and tertiary management is required. If Cut-off value<0.30, the diagnosis was low-risk MASH, which required only primary management, including lifestyle intervention and annual assessment, and liver puncture biopsy if 0.30≤Cut-off value <0.37.
